(19) United States
(12) Patent Application Publication
(10) Pub. No.: US 2017/0354802 A1
(43) Pub. Date: Dec. 14, 2017

(54) MEDICAL BALLOON

(71) Applicant: BOSTON SCIENTIFIC SCIMED, INC., Maple Grove, MN (US)

(72) Inventors: Daniel Lee Krautkremer, Plymouth, MN (US); Robert N. Squire, Maple Grove, MN (US); Jeffrey S. Lindquist, Maple Grove, MN (US); Phillip John Poisson, Maple Grove, MN (US)

(73) Assignee: BOSTON SCIENTIFIC SCIMED, INC., Maple Grove, MN (US)

(21) Appl. No.: 15/622,020

(22) Filed: Jun. 13, 2017

Related U.S. Application Data
(60) Provisional application No. 62/349,925, filed on Jun. 14, 2016.

(51) Int. Cl. A61M 25/10; A61M 25/00; B29L 31/00

(52) U.S. Cl. CPC A61M 25/104; A61M 25/1027; A61M 25/0012; B29L 2031/7543; A61M 2025/1084; B32B 2255/26

(57) ABSTRACT

An expandable medical balloon, comprising a balloon, the balloon comprising a cone portion, a waist portion and a body portion and a fiber braid disposed along the cone portion, the waist portion and the body portion of the balloon, the fiber braid comprising a first fiber and a second fiber that is different than the first fiber, the first fiber comprising a polymer material having a first melting temperature and the second fiber is a non-melting fiber.

### CROSS REFERENCE TO RELATED APPLICATIONS

This application claims the benefit of U.S. Provisional Patent Application Ser. No. 62/349,925 filed on Jun. 14, 2016, the disclosure of which is incorporated herein by reference.

### TECHNICAL FIELD

The disclosure relates to intravascular medical devices such as medical balloons, and methods of making the same.

### BACKGROUND

Medical balloons can be used to administer a variety of treatments. For example, in an angioplasty procedure, a balloon can be used to widen a constricted bodily vessel, such as a coronary artery. A balloon can also be used to deliver a tubular member, such as a stent, that is placed in the body to reinforce or to reopen a blocked vessel.

In angioplasty, the balloon can be used to treat a stenosis, or a narrowing of the bodily vessel, by collapsing the balloon and delivering it to a region of the vessel that has been narrowed to such a degree that blood flow is restricted. The balloon can be delivered to a target site by passing the catheter over an emplaced guidewire and advancing the catheter to the site. In some cases, the path to the site can be rather tortuous and/or narrow. Upon reaching the site, the balloon is then expanded, e.g., by injecting a fluid into the interior of the balloon. Expanding the balloon can expand the stenosis radially so that the vessel can permit an acceptable rate of blood flow. After use, the balloon is collapsed and withdrawn.

In stent delivery, the stent is compacted on the balloon and transported to a target site. Upon reaching the site, the balloon can be expanded to deform and to fix the stent at a predetermined position, e.g., in contact with the vessel wall. The balloon can then be collapsed and withdrawn.

Medical balloons can be manufactured by extruding a cylindrical tube of polymer and then pressurizing the tube while heating to expand the tube into the shape of a balloon. The balloon can be fastened around the exterior of a hollow catheter shaft to form a balloon catheter. The hollow interior of the balloon is in fluid communication with the hollow interior of the shaft. The shaft may be used to provide a fluid supply for inflating the balloon or a vacuum for deflating the balloon.

### BRIEF SUMMARY

This disclosure provides design, material, manufacturing method, and use alternatives for medical devices.

In one aspect, the disclosure relates to an expandable medical balloon, the expandable medical balloon comprising a balloon, the balloon comprising a cone portion, a waist portion and a body portion and a fiber braid disposed along the cone portion, the waist portion and the body portion of the balloon, the fiber braid comprising a first fiber and a second fiber that is different than the first fiber, the first fiber comprising a polymer material having a low melting temperature and the second fiber comprising a polymer material having a high melting temperature.

Alternatively or additionally to any of the embodiments above, the second fiber may have a melting temperature that is significantly higher than that of the first fiber.

Alternatively or additionally to any of the embodiments above, further comprising a first coating layer disposed between the fiber braid and an outer surface of the balloon.

Alternatively or additionally to any of the embodiments above, the first coating layer comprises a thermoplastic polyurethane.

Alternatively or additionally to any of the embodiments above, further comprising a second coating layer disposed along an outer surface of the fiber braid.

Alternatively or additionally to any of the embodiments above, the second coating layer comprises a thermoplastic polyurethane.

Alternatively or additionally to any of the embodiments above, the first fiber comprises ultra high molecular weight polyethylene.

Alternatively or additionally to any of the embodiments above, the second fiber comprises a copolyamide polymer material.

Alternatively or additionally to any of the embodiments above, the second fiber comprises a liquid crystal polymer.

Alternatively or additionally to any of the embodiments above, the second fiber comprises a liquid crystal polymer of an aromatic polyester.

Alternatively or additionally to any of the embodiments above, the balloon comprises an elastomeric polymer material.

Alternatively or additionally to any of the embodiments above, the balloon comprises poly(ether-block-amide).

Alternatively or additionally to any of the embodiments above, the first fiber has a melting temperature of about 120° C. to about 200° C.

Alternatively or additionally to any of the embodiments above, the second fiber begins to degrade at temperatures above 400° C.

Alternatively or additionally to any of the embodiments above, the second fiber begins to degrade at temperatures above 500° C.

Alternatively or additionally to any of the embodiments above, the second fiber comprises about 5% to about 50% of the total fiber cross-sectional area of the fiber braid.

In another aspect, the disclosure relates to a catheter assembly, comprising a polymeric catheter shaft, a balloon, the balloon comprising a cone portion, a waist portion and a body portion and a fiber braid disposed along the cone portion, the waist portion, and the body portion of the balloon, the fiber braid comprising a first fiber and a second fiber that is different than the first fiber, wherein an inner surface of the waist portion of the balloon is thermally bonded to an outer surface of the catheter shaft, the first fiber melts at the thermal bond at the waist portion of the balloon and the second fiber is a non-melting fiber.

Alternatively or additionally to any of the embodiments above, the first fiber has a melting temperature of about 120° C. to about 200° C.

Alternatively or additionally to any of the embodiments above, the second fiber degrades at temperatures above 400° C.

Alternatively or additionally to any of the embodiments above, the second fiber comprises about 5% to about 50% of the total fiber cross-sectional area.

In another aspect, the disclosure relates to a method of making a catheter assembly, comprising disposing a fiber braid about a balloon, the balloon comprising a cone portion, a waist portion and a body portion, the fiber braid comprising a first fiber comprising a polymer material having a low melting temperature and a second fiber that is different than the first fiber, the second fiber comprising a non-melting polymer material, disposing the balloon on a catheter shaft, and applying heat adjacent the waist portion of the balloon to thermally bond an inner surface of the fiber braid to an outer surface of the waist portion of the balloon, wherein the first fiber melts at the interface and the second fiber does not melt at the interface.

Alternatively or additionally to any of the embodiments above, applying heat adjacent to the waist portion of the balloon comprises applying heat at a temperature of about 250° C. to about 350° C.

Alternatively or additionally to any of the embodiments above, the first fiber comprises an ultra high molecular weight polyethylene.

Alternatively or additionally to any of the embodiments above, the second fiber comprises a copolyamide fiber.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example medical device;

FIG. 2 is a partial cross-sectional side view of an example medical device;

FIG. 3 is a cross-section of an example medical device taken at section 3-3 in FIG. 2;

FIG. 4 is a side view of an example medical device; and

FIG. 5 is a graph illustrating the bond tensile strength of an example medical device.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

As used herein, the terms “proximal” and “distal” refer to that which is closest to the user such as a surgeon and that which is furthest from the user respectively.

Balloons with a high burst strength may be desirable for some interventions. In some instances, polymeric sleeves may be disposed over the distal waist portion and proximal waist portion. The sleeves may have an adequate thickness to maintain balloon pressure but may result in a balloon with an increased profile. Balloons including a fiber braid of highly oriented high molecular weight polymer fibers are beneficial in that high tensile strength and burst strength can be achieved while maintaining a low balloon profile. Thermally bonded balloons made with 100% meltable fiber achieved good burst pressures. However, it may be desirable to increase the tensile strength at the interface of the melt zone.

The present disclosure relates to an expandable hybrid medical balloon exhibiting rated burst strengths of 30 atmospheres or higher, for example 30-35 atmospheres. A fiber braid is disposed on the balloon. The fiber braid includes a first fiber material that has a relatively low melting temperature and a second fiber material that has a relatively high melting temperature or is even non-melting (e.g., rather than melting, decomposition of the polymer material occurs at relatively high temperatures). The inner surface of the waist portion of the balloon may be thermally bonded to an outer surface of a catheter shaft. In at least some instances, at the thermal bond, the low melting fiber material melts, the high melting fiber material does not melt, which increases the strength of the bond at both the distal and proximal waist portions of a balloon.

The catheter assemblies using the fiber braided balloon and thermal bonds disclosed herein have proximal and distal bonds at the fiber braided balloon waist to the catheter shaft that provide improved tensile, burst and profile properties.

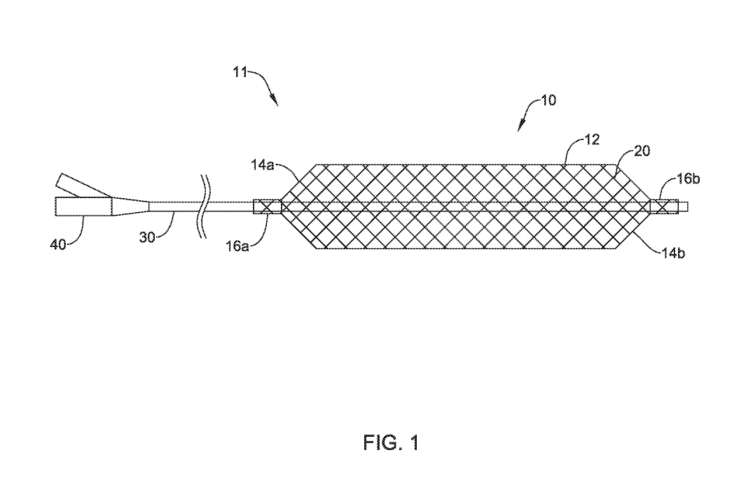

A side view of an exemplary balloon catheter 11 is illustrated in FIG. 1. The balloon catheter 11 may include an expandable medical balloon 10 mounted on the distal end of a catheter shaft 30. A fiber braid 20 may be disposed along the outer surface of the balloon 10. Catheter shaft 30 extends from a manifold assembly 40 at a proximal end of the catheter shaft 30. The balloon 10 is shown having a body portion 12, a proximal cone portion 14a, a distal cone portion 14b, a proximal waist portion 16a, and a distal waist portion 16b. The balloon 10 may be secured to the catheter shaft 30 at the proximal waist portion 16a and the distal waist portions 16b, respectively.

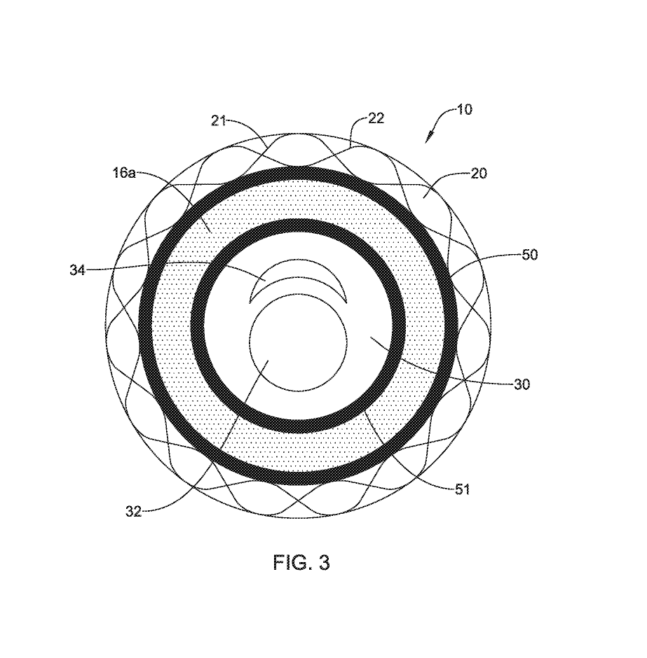

For the balloon catheter 11 shown in FIG. 1, the catheter shaft 30 is depicted as a dual-lumen catheter shaft 30 that includes a guidewire lumen 32 for a guidewire (not shown) and an inflation lumen 34 for inflation of the balloon 10 as shown in cross-section in FIG. 3. Alternatively, the catheter shaft 30 may include an inner tubular member defining the guidewire lumen 32 and an outer tubular member extending around the inner tubular member. In these instances, the inflation lumen 34 may be defined between the inner tubular member and the outer tubular member. In such cases, the proximal waist portion 16a may be secured to a distal end region of the outer tubular member and the distal waist portion 16b may be secured to a distal end region of the inner tubular member. Other catheter shafts are contemplated.

The balloon may be preformed, for instance by radial expansion of a tubular parison, which is optionally also longitudinally stretched. The extruded parison may be radially expanded as is into a mold or by free-blowing. Alternatively, the parison may be pre-stretched longitudinally before expansion or reformed in various ways to reduce thickness of the balloon cone and waist regions prior to radial expansion. The blowing process may utilize pressurization under tension, followed by rapid dipping into a heated fluid; a sequential dipping with differing pressurization; a pulsed pressurization with compressible or incompressible fluid, after the material has been heated. Heating may also be accomplished by heating the pressurization fluid injected into the parison. Balloon diameters range from 4 mm to 26 mm depending on the application.

The balloon 10 may be formed of a suitable material which may be made by radial expansion of a tubular parison, typically thermoplastic polymers. The balloon 10 may be formed from typical balloon materials including compliant, semi-compliant and non-compliant balloon materials. These materials include both elastomers and non-elastomers. For example, the balloon catheter may be formed from a compliant material such as poly(ether-block-amide), or a non-compliant material such as nylon, or combinations thereof. Exemplary materials are discussed in more detail below. A coating (not shown) may be disposed on balloon 10 prior to application of fiber braid 20.

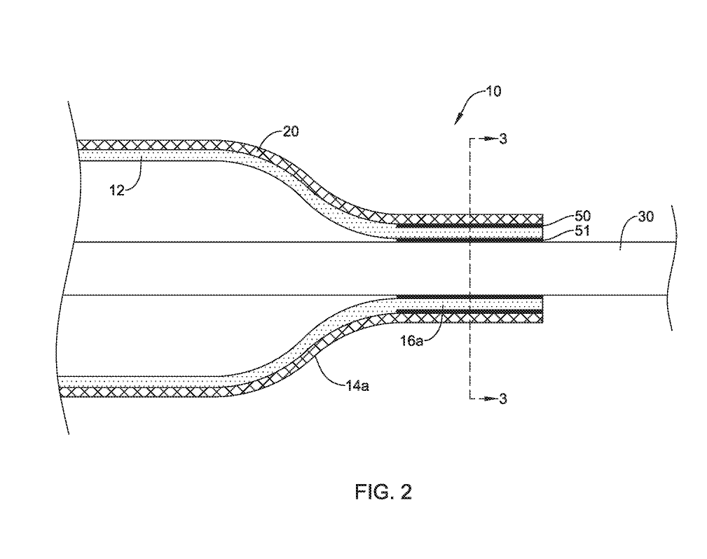

FIG. 2 is a partial cross-section of the balloon 10 disposed on the distal portion of the catheter shaft 30 wherein the fiber braid 20 is bonded (e.g., thermally bonded, adhesively bonded, etc.) to an outer surface of the proximal waist portion 16a at a bond 50 and an inner surface of the proximal waist portion 16a is bonded (e.g., thermally bonded, adhesively bonded, etc.) to an outer surface of a distal portion of the catheter shaft 30 at a bond 51. This is also illustrated in cross-section in FIG. 3 which is taken at section 3-3 from FIG. 2. The distal waist portion 16b may also be secured to the catheter shaft 30 with a thermal bond. A coating (not shown) may be disposed along the exterior of the fiber braid 20. In an example, the bond 50 includes a mixture of the elastomeric material of the balloon 10 and the fiber braid 20. The bond 51, for example, can include only the elastomeric material of the balloon 10 bonded to the catheter shaft 30. In an example, bonds 50 and 51 could form a single bond, bonding the balloon 10 to the distal portion of the catheter shaft 30.

The fiber braid 20 includes at least one first fiber 21 and at least one second fiber 22 that is different than the first fiber 21. The first fiber 21 comprises a relatively low melting temperature polymer material, for example, polymer materials having a melting point of about 120° C. to about 200° C. The second fiber 22 comprises a relatively high melting temperature polymer material or a non-melting polymer material. Such polymer materials have melting points of about 300° C. or higher, or for non-melting polymer materials, decompose (e.g., rather than melt) at temperatures of about 400° C. or higher, or 500° C. or higher. The fiber braid 20 may be disposed along the waists, cones and body portion of the balloon, or at least a portion thereof. The bond 50 may include partially melted fiber braid 20, such as, for example the first fiber 21 may be melted and the second fiber 22 may remain unmelted.

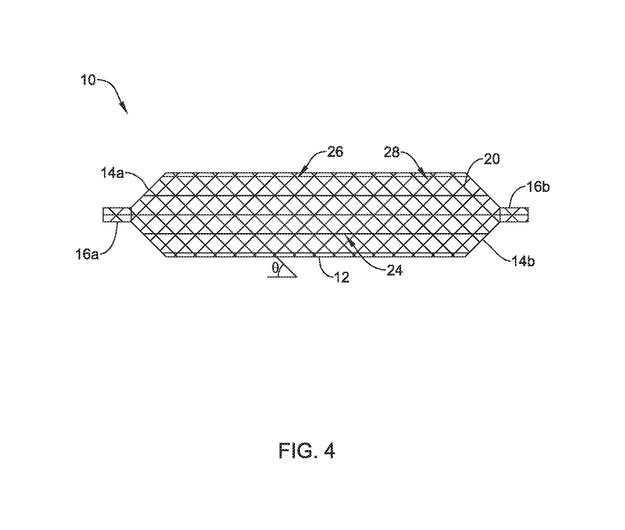

FIG. 4 is a side view of an embodiment of an exemplary expandable medical balloon 10 illustrating a fiber braid 20 having an exemplary braid pattern. In this embodiment, the fiber braid 20 includes longitudinal strands 24, radial strands 26 and crossing radial strands 28. The radial strands 26 cross the longitudinal strands 24 at a crossing angle θ and the crossing radial strands 28 cross the longitudinal strands 24 at an angle that may be supplementary to the radial strands 26 along the balloon 10. For example, crossing angles may be from about 25° to about 75°, or about 40° to about 75°, or about 50° to about 75° or about 50° to about 65°. However, other angles that are contemplated include angles that vary amongst groups of strands. The fiber braid includes intermingled first fiber 21 and second fiber 22 as described above (not depicted in FIG. 4).

In some embodiments, the fiber braid 20 may include a number of different longitudinal strands 24, radial strands 26, and/or crossing strands 28. For example, the fiber braid 20 may include 10-24, or about 12-20, or about 14-18, or about 16 longitudinal strands 24. In some of these and in other instances, the fiber braid 20 may include 24-48, or about 28-40, or about 30-34, or about 32 radial strands 26. In some of these and in other instances, the fiber braid 20 may include 25-48, or about 28-40, or about 30-34, or about 32 radial strands 28. For example, in some embodiments, the braider includes 32-48 radial carriers and 16-24 longitudinal carriers having fiber bobbins on the radial carriers and/or longitudinal carriers spooled with 2 strands per bobbin or 1 strand per bobbin. These are just examples. Other numbers of strands are contemplated.

In some embodiments, the longitudinal fiber strands may include 8 high or non-melting fiber strands and 8 low melting point fiber strands. In other words, the number of non-melting and low melting strands may be balanced or otherwise be the same. In other instances, differing numbers of non-melting and low melting point strands. The radial strands may include from 0 to 16 high or non-melting fiber strands and from 16 to 32 low melting point fiber strands. Again, variations are contemplated.

The shape, form and the configuration of the fibers may vary. For example, the fibers may take on different cross-sectional shapes, for example, circular, elliptical or spherical, flat, or some combination thereof. The number of fibers can also vary. In some instances, an individual fiber may include a single filament, whereas in other instances two, three, four, five, or more filaments may comprise a single fiber. In some instances, the pattern and/or crossing angles for the fibers may be varied and can be uniform, non-uniform or some combination thereof. The fiber coverage or density on the balloon may also be varied.

Suitable fiber materials include, but are not limited to, polyesters, polyolefins, polyamides, polyurethanes, liquid crystal polymers, polyimides, and mixtures thereof. For example, the first fiber 21 may include an ultra high molecular weight polyethylene, and the second fiber 22 may include a liquid crystal polymer, or a co-polyamide. Suitable fiber materials are discussed in more detail below.

The balloon 10 may be capable of being inflated to relatively high pressures. For example, the balloon 10 may be inflated to pressures up to about 20 atm or more, or up to about 25 atm or more, or up to about 30 atm or more, or up to about 40 atm or more, or up to about 45 atm or more, or up to about 50 atm or more, or about 20-50 atm, or about 25-40 atm, or about 30-50 atm. At such elevated pressures, the bond between the proximal waist portion 16a and the catheter shaft 30 (as well as the bond between the distal waist portion 16b and the catheter shaft 30) is maintained. Furthermore, the bond between the fiber braid 20 and the balloon 10 is also maintained at these elevated pressures.

As noted above, the balloon 10 may be formed from a variety of suitable materials known in the art. Such materials may include, but are not limited to, low, linear low, medium and high density polyethylenes, polypropylenes and copolymers and terpolymers thereof; polyurethanes; polyesters and copolyesters; polycarbonates; polyamides; thermoplastic polyimides; polyetherimides; polyetheretherketones (PEEK) and PES (polyether sulfone); and copolymers and terpolymers thereof. Physical blends and copolymers of such materials may also be used.

Examples of polyesters include, but are not limited to, polyethylene terephthalate (PET), polyethylene naphthalate (PEN), polybutylene terephthalate, and copolymers thereof. Examples of polyamides which may be used include nylon 6, nylon 64, nylon 66, nylon 610, nylon 610, nylon 612, nylon 46, nylon 9, nylon 10, nylon 11, nylon 12, and mixtures thereof. Examples of suitable polyurethanes include, but are not limited to, aromatic polyether-based thermoplastic polyurethanes (TPUs) such as those available under the tradename of Tecothane® from Thermedics; Tecoflex® thermoplastic polyurethanes commercially available from Lubrizol Corporation in Wickliffe, Ohio; thermoplastic polyurethane elastomer available under the tradename of Pellethane®, such as Pellethane® 2363-75D from Dow Chemical Co.; and high strength engineering thermoplastic polyurethane available under the tradename of Isoplast®, such as Isoplast® 301 and 302 available from Dow Chemical Co.

In some embodiments, balloon 10 may be formed from poly(ether-block-amide) copolymers. The polyamide/polyether block copolymers are commonly identified by the acronym PEBA (polyether block amide). The polyamide and polyether segments of these block copolymers may be linked through amide linkages, for example, some are ester linked segmented polymers, e.g., polyamide/polyether polyesters. Such polyamide/polyether/polyester block copolymers are made by a molten state polycondensation reaction of a dicarboxylic polyamide and a polyether diol. The result is a short chain polyester made up of blocks of polyamide and polyether. Polymers of this type are commercially available under the tradename of Pebax® from Arkema. Specific examples are the “33” series polymers with hardness 60 and above, Shore D scale, for example, Pebax® 6333, 7033 and 7233. These polymers are made up of nylon 12 segments and poly(tetramethylene ether) segments linked by ester groups.

Polyester/polyether segmented block copolymers may also be employed herein. Such polymers are made up of at least two polyester and at least two polyether segments. The polyether segments are the same as previously described for the polyamide/polyether block copolymers useful in the disclosure. The polyester segments are polyesters of an aromatic dicarboxylic acid and a two to four carbon diol.

In some embodiments, the polyether segments of the polyester/polyether segmented block copolymers are aliphatic polyethers having at least 2 and no more than 10 linear saturated aliphatic carbon atoms between ether linkages. The ether segments may have 4-6 carbons between ether linkages, and they may include poly(tetramethylene ether) segments. Examples of other polyethers which may be employed in place of the tetramethylene ether segments include polyethylene glycol, polypropylene glycol, poly(pentamethylene ether) and poly(hexamethylene ether). The hydrocarbon portions of the polyether may be optionally branched. An example is the polyether of 2-ethylhexane diol. Generally such branches will contain no more than two carbon atoms. The molecular weight of the polyether segments is suitably between about 400 and 2,500, and more suitably between 650 and 1000.

In some embodiments, the polyester segments of the polyester/polyether segmented block copolymers are polyesters of an aromatic dicarboxylic acid and a two to four carbon diol. Suitable dicarboxylic acids used to prepare the polyester segments of the polyester/polyether block copolymers are ortho-, meta- or para-phthalic acid, napthalenedicarboxylic acid or meta-terphenyl-4,4′-dicarboxylic acids. Specific examples of polyester/polyether block copolymers are poly(butylene terephthalate)-block-poly(tetramethylene oxide) polymers such as Arnitel® EM 740, sold by DSM Engineering Plastics, and Hytrel® polymers, sold by DuPont, such as Hytrel® 8230.

As noted above, the fiber braid 20 may also be formed from a variety of suitable materials. Some specific examples include, but are not limited to, polyesters such as polyethylene terephthalate (PET), polybutylene terephthalate (PBT), and polytrimethylene terephthalate (PTT). Polyamides include nylons and aramids such as Kevlar®. Polyolefins include ultrahigh molecular weight polyethylene, and very high density polyethylene, and polypropylene fibers. Elastomeric fibers can be used in some cases. In some specific embodiments of the disclosure, fibers that are high strength materials may also be suitable in some applications.

In some embodiments, the first fiber 21 comprises an ultra high molecular weight polyethylene (UHMPE). Commercially available UHMPEs include, but are not limited to, Dyneema® fiber available from DSM Dyneema BVm Heerlen, Netherlands, Spectra® fiber available from Honeywell in Morristown and Pegasus UHMWPE fiber available from Pegasus Materials in Shanghai, China.

In some embodiments, the first fiber 21 is a high melting temperature fiber, such as a liquid crystal polymer, for example, Vectran®, an aromatic polyester available from Kuraray Ltd. In Tokyo, Japan. In some embodiments, the liquid crystal polymer is formed by the polycondensation of 4-hydroxybenzoic acid and 6-hydroxynaphthalene-2-carboxylic acid.

The UHMWPE fibers provide excellent strength and modulus with a small filament size to provide excellent balloon coverage and maintaining a minimal profile. However, when melted, the fibers lose their high molecular orientation and consequently, bond tensile strength at the proximal waist portion 16a and/or the distal waist portion 16b of the balloon at the thermal bond interface may decrease.

In some embodiments, the second fiber 22 comprises a copolyamide, for example, Aramid fiber. Aramid fiber are aromatic polyamides and can be classified as heat-resistant, non-melting fibers wherein degradation starts from 500° C. Typically, aramids are long-chain polyamides wherein at least 85% of the amide linkages are attached to two aromatic rings. Many of these materials are classified as having no melting point. One commercially available aramid fiber is Technora®, para-aramid which is a polyamide copolymer. Technora® fiber is available from Teijin Aramid, a subsidiary of the Teijin Group in the United Kingdom. Other examples of suitable aramid fibers include, but are not limited to, Kevlar® fiber available from DuPont in Wilmington, Del., Nomex® meta-aramid fiber also available from DuPont, and Twaron fiber which is also available from Teijin Aramid.

The high or non-melting fibers do not melt during thermal welding and thus maintain a high tensile load at the proximal and distal waist of the balloon at the thermal bond interface.

The second fiber 22 may be employed in amounts of about 5% to about 50% of the total fiber cross-sectional area, and suitably about 10% to about 40% or 15% to about 35%. It has been found that increasing the amount of the second fiber 22 increases tensile strength.

Additionally coatings may be optionally applied to the balloon including between the outer surface of the balloon and the braid, over the outer surface of the braid or both. In some embodiments, the coating includes a thermoplastic elastomer. In some embodiments, the coating includes a thermoplastic polyurethane. In some embodiments, the coating of thermoplastic polyurethane is applied to the balloon prior to braiding and is also applied to the balloon/braid after braiding.

The hybrid balloons having the mixed fiber strands exhibited improved maximum load proximal bond tensile strength of between about 10 to about 15 lbf.

The catheter shaft 30 may be formed from any suitable shaft material. Examples include, but are not limited to, polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the shaft material mixture can contain up to about 6 percent LCP. In some embodiments, the catheter shaft 30 is formed from a polyamide, for example Grilamid® which is commercially available from EMS-Grivory.

The above lists are intended for illustrative purposes only, and not as a limitation on the present disclosure. It is within purview of those of ordinary skill in the art to select other polymers without departing from the scope of this disclosure.

### Examples

In at least some instances, a catheter assembly may be formed by bonding an inner and an outer catheter shaft assembly having a dual lumen shaft formed from Grilamid®. A balloon parison (tubular member) formed of Pebax® 7033 may be stretched, placed in a balloon mold and formed by radial expansion. The resultant balloon was 8×100 mm. The tubes may alternatively have 4 mm or 12 mm diameters. A mandrel was installed and the balloon was inflated to 13 psi. The balloon was plasma treated with oxygen, and dip coated with 2.5% solids Lubrizol SG 60D thermoplastic polyurethane in a cosolvent blend of 50% toluene/50% tetrahydrofuran. The plasma treatment was conducted in a Nordson-March RF Plasma Chamber at a 100 sccm O₂ flow rate, base pressure 100 mtorr, 250 watts, 90 seconds times four cycles. The coating thickness was 4 μm. The dipping process may take up to four repeat cycles to achieve the desired thickness with 10 minutes in between each cycle at a dip down and up speed of 50 in./min. with a hold time of 2 seconds in a 100 ml graduated cylinder.

The balloon was then braided with an ultra high molecular weight, highly oriented Pegasus polyethylene (UHMWPE) fibers and Technora® Aramid fibers consisting of 16 longitudinal and 32 radial fiber strands. Longitudinally, the balloons were braided with 8 Technora® Aramid fiber strands and 8 Pegasus UHMWPE fiber strands. Radially, the balloons were braided with 0, 8 and 16 Technora® Aramid fiber strands and 32, 24 and 16 Pegasus UHMWPE fiber strands respectively. The braider included 32 radial carriers and 16 longitudinal carriers having fiber bobbins on the radial carriers spooled with 2 strands per bobbin or 1 strand per bobbin.

The following table, Table 1, illustrates the fiber density calculations:

TABLE 1

| | | | | | | | |
|---|---|---|---|---|---|---|---|
| | | | | # Filaments/ | Filament | Total # | Cross Sectional Area of |
| Section | Fiber | # carriers | # strands | strand | diameter (um) | Filaments | Total Filaments (um) |

16 Technora Radial Strands (8/8 for long)

| | | | | | | | |
|---|---|---|---|---|---|---|---|
| Long | Pegasus 33d | 8 | 1 | 16 | 17 | 128 | 29,053 |
| Long | Technora 62d | 8 | 1 | 25 | 12 | 200 | 22,619 |
| Radial | Pegasus 33d | 16 | 2 | 16 | 17 | 512 | 116,214 |
| Radial | Technora 62d | 16 | 1 | 25 | 12 | 400 | 45,239 |
| | | | | | | Pegasus 33d | 145,267 |
| | | | | | | Technora 62d | 67,858 |
| | | | | | | % Pegasus | 68% |
| | | | | | | % Technora | 32% |
| | | | | | | (by area) | |

0 Technora Radial Strands (8/8 for long)

| | | | | | | | |
|---|---|---|---|---|---|---|---|
| Long | Pegasus 33d | 8 | 1 | 16 | 17 | 128 | 29,053 |
| Long | Technora 62d | 8 | 1 | 25 | 12 | 200 | 22,619 |
| Radial | Pegasus 33d | 32 | 2 | 16 | 17 | 1024 | 232,428 |
| Radial | Technora 62d | 0 | 1 | 25 | 12 | 0 | — |
| | | | | | | Pegasus 33d | 261,481 |
| | | | | | | Technora 62d | 22,619 |
| | | | | | | % Pegasus | 92% |
| | | | | | | % Technora | 8% |
| | | | | | | (by area) | |

The balloons were then again plasma treated and dip coated in 50:50 toluene: THF solvent with 2.5% solids Lubrizol SG 60D TPU to a thickness of 4 μm. The proximal and distal balloon waists were trimmed, and the balloon was installed onto the inner and outer shaft assembly. A hot jaw bond at 260° C. was applied to the proximal balloon waist for 25 seconds. The jaw hole was 0.075 in. wide and the jaw hole ID was 0.100 in. For the distal waist, the hot jaw bond was at 260° C. for 15 seconds and hot jaw hole ID was 0.093 in. and the jaw width was 0.2 in.

The proximal bond tensile strength was tested for each sample balloon. The tensile strength of the proximal balloon bond without the inner shaft was tested with an Instron testing apparatus at a gage length of 1 in. and a speed of 20 in./min. Maximum load was recorded. The test is American National Standards Institute (ANSI) approved. The balloon is optionally inflated to 2 atmospheres, deflated and flattened for testing. The catheter shaft was cut approximately 2.5 in. from the proximal balloon bond and the balloon was circumferentially cut to a minimum length of 0.6 in. from the cone/body transition. Balloons can be cut longer to provide more grip area. The inner shaft is then removed and each end of the balloon is placed in a grip with the shaft being in the lower grip, the balloon in the upper grip and the proximal bond is centered in the gauge length. The Instron is then started and the tensile strength recorded.

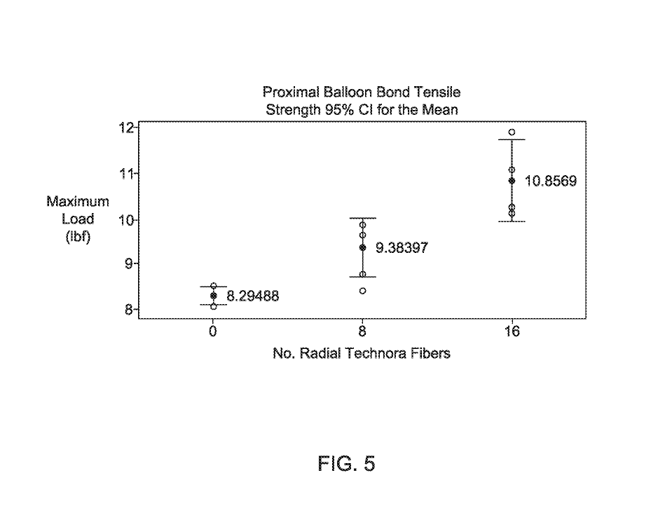

FIG. 5 is a graph illustrating the bond tensile strength for balloons having 8 longitudinal Technora® fiber strands and 8 longitudinal UHMWPE fiber strands, and 0, 8, and 16 Technora® radial strands and 32, 24, and 16 UHMWPE fiber strands respectively. Bond tensile strength increases with an increasing number of radial strands of Technora® fibers. The braider included 32 radial carriers and 16 longitudinal carriers having fiber bobbins on the radial carriers spooled with 2 strands per bobbin or 1 strand per bobbin. Average tensile failure for balloons having only UHMWPE fiber strands were found to have an average tensile load failure of about 6-7 lbf.

It was further determined that employing UHMWPE fiber strands in combination with Technora® fiber strands, resulted in the UHMWPE fibers forming a melt pool at the thermal bond that provides a strong attachment for the non-melted Technora® fibers. The UHMWPE fibers provide a high strength balloon body due to the high molecular orientation of the UHMWPE fibers and when melted at the proximal and distal balloon waist, they provide a secure attachment for the non-melted Technora® fiber strands.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

1. An expandable medical balloon, comprising:
a balloon, the balloon comprising a cone portion, a waist portion, and a body portion; and
a fiber braid disposed along the cone portion, the waist portion, and the body portion of the balloon, the fiber braid comprising a first fiber and a second fiber that is different than the first fiber, the first fiber comprising a polymer material having a first melting temperature and the second fiber comprising a polymer material having a second melting temperature different than the first melting temperature.

2. The expandable medical balloon of claim 1, further comprising a first coating layer disposed between the fiber braid and an outer surface of the balloon.

3. The expandable medical balloon of claim 2, wherein the first coating layer comprises a thermoplastic polyurethane.

4. The expandable medical balloon of claim 1, further comprising a second coating layer disposed along an outer surface of the fiber braid.

5. The expandable medical balloon of claim 4, wherein the second coating layer comprises a thermoplastic polyurethane.

6. The expandable medical balloon of claim 1, wherein the first fiber comprises ultra high molecular weight polyethylene.

7. The expandable medical balloon of claim 1, wherein the second fiber comprises a copolyamide polymer material.

8. The expandable medical balloon of claim 1, wherein the balloon comprises an elastomeric polymer material.

9. The expandable medical balloon of claim 1, wherein the balloon comprises poly(ether-block-amide).

10. The expandable medical balloon of claim 1, wherein the first melting temperature is from about 120° C. to about 200° C.

11. The expandable medical balloon of claim 1, wherein the second fiber begins to degrade at temperatures above 400° C.

12. The expandable medical balloon of claim 1, wherein the second fiber comprises about 5% to about 50% of the total fiber cross-sectional area of the fiber braid.

13. A catheter assembly, comprising:
a polymeric catheter shaft;
a balloon, the balloon comprising a cone portion, a waist portion, and a body portion; and
a fiber braid disposed along the cone portion, the waist portion, and the body portion of the balloon, the fiber braid comprising a first fiber and a second fiber that is different than the first fiber;
wherein an inner surface of the waist portion of the balloon is thermally bonded to an outer surface of the catheter shaft, the first fiber melts at the thermal bond at the waist portion of the balloon and the second fiber is a non-melting fiber.

14. The catheter assembly of claim 13, wherein the first fiber has a melting temperature of about 120° C. to about 200° C.

15. The catheter assembly of claim 13, wherein the second fiber degrades at temperatures above 400° C.

16. The catheter assembly of claim 13, wherein the second fiber comprises about 5% to about 50% of the total fiber cross-sectional area.

17. A method of making a catheter assembly, comprising:
disposing a fiber braid about a balloon, the balloon comprising a cone portion, a waist portion, and a body portion, the fiber braid comprising a first fiber comprising a polymer material having a first melting temperature and a second fiber that is different than the first fiber, the second fiber comprising a non-melting polymer material;
disposing the balloon on a catheter shaft; and
applying heat adjacent to the waist portion of the balloon to thermally bond an inner surface of the fiber braid to an outer surface of the waist portion of the balloon at an interface of the waist portion of the balloon and the fiber braid, wherein the first fiber melts at the interface and the second fiber does not melt at the interface.

18. The method of claim 17, wherein applying heat adjacent to the waist portion comprises applying heat at a temperature of about 250° C. to about 350° C.

19. The method of claim 17, wherein the first fiber comprises an ultra high molecular weight polyethylene.

20. The method of claim 17, wherein the second fiber comprises a copolyamide fiber.
